(19) United States
(12) Patent Application Publication
(10) Pub. No.: US 2020/0397795 A1
(43) Pub. Date: Dec. 24, 2020

(54) PROPHYLACTIC TREATMENT OF VENOUS THROMBOEMBOLISM

(71) Applicant: JANSSEN PHARMACEUTICALS, INC., Titusville, NJ (US)

(72) Inventors: Elliot Stephen Barnathan, Havertown, PA (US); Concetta Lipardi, Princeton, NJ (US)

(21) Appl. No.: 16/907,119

(22) Filed: Jun. 19, 2020

Related U.S. Application Data
(60) Provisional application No. 62/992,320, filed on Mar. 20, 2020.
(60) Provisional application No. 62/864,654, filed on Jun. 21, 2019.

(51) Int. Cl. A61K 31/5377; A61K 9/00; A61P 7/02

(52) U.S. Cl. CPC A61K 31/5377; A61P 7/02; A61K 9/0053

(57) ABSTRACT

Method of prophylactic treatment of acutely ill medical patients with rivaroxaban to reduce venous thromboembolism (VTE) and VTE-related death during hospitalization and post-hospital discharge.

### CROSS REFERENCE TO RELATED APPLICATIONS

This application claims the benefit of U.S. Provisional Application 62/992,320, filed on Mar. 20, 2020 and U.S. Provisional Application 62/864,654, filed Jun. 21, 2019. The entire contents of these applications are incorporated herein by reference in their entirety.

### FIELD OF INVENTION

The present invention concerns treatment against venous thromboembolism (VTE) and VTE-related death in patients with an acute medical illness.

### BACKGROUND OF INVENTION

Between the United States and most European countries, there are an estimated 20 million acutely ill, hospitalized medical patients annually. Approximately one quarter of these acutely ill patients develop a blood clot, resulting in death for approximately 15% of the blood clot patients. Venous thromboembolism (VTE) is a leading cause of premature death and major morbidity throughout the world, and approximately 75% of all autopsy-documented fatal pulmonary embolic (PE) events in hospitalized patients occur in medically ill patients.

The hospitalized, acutely ill medical patients remain at high risk for development of VTE during hospitalization and post-discharge; however, it is estimated that less than 4% of the U.S. patients receive post-discharge thromboprophylaxis. While there exists antithrombotic guidelines recommending thromboprophylaxis for hospitalized medical patients at increased risk of VTE until the patient is fully mobile or discharged, studies suggest VTE risk extends far beyond discharge, with approximately 80% of VTE events occurring within 6 weeks of hospital discharge. Previous trials of extended thromboprophylaxis (i.e., beyond hospital discharge) with low molecular weight heparin or direct oral anticoagulants (DOACs) have demonstrated prevention of VTE events. However, this was associated with an increase in the risk of bleeding events. While previous attempts at extended thromboprophylaxis (beyond hospital discharge) demonstrated some prevention of VTE events, these attempts were associated with increased risks of bleeding events, which has led to antithrombotic guidelines advising against routine use of extended thromboprophylaxis in acutely ill medical patients.

There remains a need for optimizing the benefit-risk of extended thromboprophylaxis in acutely ill medical patients. Optimization should be focused on improved patient selection defining at-risk groups that would benefit from extended thromboprophylaxis and excluding patients at high risk of bleeding.

### SUMMARY

The invention concerns the use of rivaroxaban (5-chloro-N-({(5S)-2-oxo-3-[4-(3-oxo-4-morpholinyl)phenyl]-1,3,oxazolidin-5-yl}methyl)-2-thiophenecarboxamide) in a prophylactic treatment against VTE and VTE-related death during hospitalization and post-hospital discharge in adult patients admitted for an acute medical illness and at risk for thromboembolic complications due to moderate or severe restricted mobility and other risks factors for VTE for a defined population of human patients. The invention also concerns the prophylactic treatment of adult human patients with rivaroxaban and products of rivaroxaban in an acutely ill medical population at risk of venous thromboembolic complications and the population is at lower risk of bleeding.

In one embodiment, the invention is a method of prophylactic treatment against venous thromboembolism (VTE) and VTE-related death during hospitalization and post-hospital discharge in an adult human patient admitted for an acute medical illness and at risk for thromboembolic complications due to moderate or severe restricted mobility and other risks factors for VTE, by administering to the adult human patient rivaroxaban in an amount effective in prophylactically treating against VTE and VTE-related death during hospitalization and post-hospital discharge in an adult human patient admitted for an acute medical illness and at risk for thromboembolic complications due to moderate or severe restricted mobility and other risks factors for VTE, wherein rivaroxaban is administered in an amount of 10 mg once daily. In certain embodiments, the invention concerns oral formulations of rivaroxaban, including 10 mg oral formulations. In various embodiments, the products of the invention may be administered to an adult human patient with or without food.

Another embodiment of the invention is the method of prophylactic treatment against venous thromboembolism (VTE) and VTE-related death during hospitalization and post-hospital discharge in an adult human patient admitted for an acute medical illness and at risk for thromboembolic complications due to moderate or severe restricted mobility and other risks factors for VTE, by administering to the adult human patient a product comprising rivaroxaban in an amount effective in prophylactically treating against VTE and VTE-related death during hospitalization and post-hospital discharge in an adult human patient admitted for an acute medical illness and at risk for thromboembolic complications due to moderate or severe restricted mobility and other risks factors for VTE, wherein rivaroxaban is administered in an amount of 10 mg once daily. In certain embodiments, the product is an oral formulation of rivaroxaban, including an oral formulation of 10 mg of rivaroxaban.

In another embodiment, the methods of the invention are to prophylactic treatment against VTE and VTE-related death during hospitalization and post-hospital discharge in an adult human patient admitted for an acute medical illness, wherein the acute medical illness includes, but not limited to, heart failure, active cancer, acute ischemic stroke, acute infectious and inflammatory disease and acute respiratory insufficiency. Also, in certain other embodiments, the methods of the present invention concern other risks factors for VTE, which include, but not limited to, prolonged immobilization, age 75 or older, history of cancer, history of VTE, history of heart failure, thrombophilia, acute infectious disease contributing to hospitalization, and body mass index (BMI) equal to or greater than 35 kg/m².

Another embodiment of the present invention is defining a patient population based on acute medical illnesses as discussed and defined herein, and assessing risks factors for VTE as discussed and defined herein, and defining patient population based on risk level of bleeding. In certain embodiments of the invention, the risk factors for high risk of bleeding can include active cancer (e.g., undergoing acute, in-hospital treatment), medical history of bronchiectasis/pulmonary cavitation, dual antiplatelet therapy at baseline, active gastroduodenal ulcer, and any bleeding in the previous three months prior to hospitalization. In another embodiment of the invention, treatment for prophylaxis against VTE and VTE-related death only begins after it has been determined that the adult human patient is not at a high risk of bleeding as defined and discussed herein.

In certain embodiments of the present invention, the methods for prophylactic treatment against VTE and VTE-related death in adult patients, as defined and discussed herein, may begin up to 72 hours after admittance to the hospital for an acute medical illness defined and discussed herein. In other embodiments, the methods for prophylactic treatment against VTE and VTE-related death in adult patients may begin at any point during hospitalization and post-hospital discharge in adult patients admitted for an acute medical illness and at risk for thromboembolic complications due to moderate or severe restricted mobility and other risks factors for VTE for a defined population of human patients. Another embodiment of the present invention is the step of beginning the prophylactic treatment against VTE and VTE-related death during hospitalization and post-hospital discharge in adult patients admitted for an acute medical illness and at risk for thromboembolic complications due to moderate or severe restricted mobility and other risks factors for VTE upon assessing that the adult human patient is in a population of human patients as defined and discussed herein. In another embodiment, the methods of treatment begin on determining an adult human patient is within a population of patients as defined and discussed herein, beginning prophylactic treatment and continuing prophylactic treatment throughout hospitalization and extending prophylactic treatment for a time necessary for the adult human patient to be at an acceptable low level risk of VTE or VTE-related death. In certain embodiments, the duration of treatment (hospitalized and post-hospital discharge) may include 31-39 days.

### DETAILED DESCRIPTION OF THE INVENTION

A 10 mg dose once daily of rivaroxaban has been shown to be effective in extended prophylactic treatment against VTE and VTE-related deaths in adult patients with acute medical illness who are at a low risk of bleeding. Once daily doses of 10 mg rivaroxaban may be given orally. While rivaroxaban is commercially available (i.e., XARELTO®), preparation methods and formulations of rivaroxaban are known in the art, with preparation disclosed in WO 01/47919, for example, and formulations disclosed in U.S. Pat. No. 7,157,456 and to U.S. Pat. No. 9,402,851. The present invention provides for effective pharmaceutical preparations/products that, besides non-toxic, inert pharmaceutically suitable excipients and/or carriers, comprise rivaroxaban.

As provided herein, the present invention has shown unexpected results in extended prophylactic treatment against VTE and VTE-related deaths in adult human patients with acute medical illness and at low risk of bleeding for rivaroxaban in 10 mg once daily dose. The specific adult human patient population was defined through multiple studies focused on extended prophylactic treatment, including treatment during hospitalization and post-hospitalization discharge for prescribed periods of time. The studies discussed herein combined to provide safer, more effective adult patient selection for the methods of prophylactic treatment against VTE and VTE-related deaths in acute medically ill adult human patients at low risk of bleeding.

In an embodiment directed to reducing the risk of venous thromboembolism (VTE) and VTE-related death in adult human patients with acute medical illness as defined herein and reduced risk of bleeding, the term “safe” refers to a dosage shown to be acceptable for administration to reduce the risk of venous thromboembolism (VTE) and VTE-related death, such as a dosage shown in trials of over nearly 10,000 patients taking rivaroxaban. “Safe” and “safer” for the methods disclosed herein may also be measured by the net clinical benefit of the inventive method or product compared to administration of enoxaparin. The net clinical benefit outcome consists of venous thromboembolism (VTE) and VTE-related death, major bleeding and non-major clinically relevant bleeding.

According to the invention, the terms “effective” or “efficacy,” as they relate to terms such as dose, dosage regimen, or treatment with rivaroxaban refer to reducing venous thromboembolism (VTE) and VTE-related death as compared to enoxaparin or a placebo; and the terms refer to a dosage shown to be acceptable for administration to reduce the risk of venous thromboembolism (VTE) and VTE-related death in patients with acute medical illness and low risk of bleeding.

According to certain embodiments of the present methods, the acute medical illness comprises Coronavirus disease (COVID-19). COVID-19 is an acute infectious disease caused by the severe acute respiratory syndrome coronavirus 2 (SARS-CoV-2). Symptoms of COVID-19 may include flu-like symptoms such as fever, sore throat, cough, and respiratory/breathing difficulties. The symptoms may be mild, or in some cases more severe, for example leading to pneumonia, acute respiratory distress syndrome, disseminated intravascular coagulation, which often require hospitalization and intensive care. In certain cases the disease is fatal.

Embodiments of the invention provide methods for prophylactic treatment against venous thromboembolism (VTE) and VTE-related death for hospitalized adult patients infected with COVID-19. As understood through certain aspects of the invention, hospitalized adult patients infected with COVID-19 who have an elevated D-Dimer level are at increased risk of developing venous thromboembolism, pulmonary embolism, adult respiratory distress syndrome (ARDS), disseminated intravascular coagulation (DIC) and death. Therefore, prophylactic treatment of such patients via the present methods is provided.

In certain embodiments, the acute medical illness comprises acute infections and inflammatory diseases, including acute infectious diseases, including but not limited to, COVID-19 infection. In certain embodiments, the present methods concern other risks factors for VTE, which include, but are not limited to, acute infections contributing to hospitalization, including, but not limited to, COVID-19 infection.

Study 1: Magellan

The objective of Study 1 (MAGELLAN or MAGELLAN Study) was to compare efficacy and safety of venous thromboembolism (VTE) prophylaxis with oral rivaroxaban 10 mg once daily administered for 35±4 days (hereafter referred to as 35 days or Day 35) to subcutaneous enoxaparin 40 mg once daily (OD) administered for 10±4 days (hereafter referred to as 10 days or Day 10) in men and women aged 40 years or above, who have been hospitalized for a medical illness.

Diagnosis and main criteria for inclusion in Study 1 was men and women 40 years or above and at risk for VTE being hospitalized for acute medical illness conditions, including: heart failure (New York Heart Association [NYHA] class III or IV); active cancer (e.g., admitted for chemotherapy or for the treatment of a complication of active cancer); acute ischemic stroke; acute infectious and inflammatory diseases, including acute rheumatic diseases; and acute respiratory insufficiency. Also at least one additional risk factor for VTE, including: severe varicosis (varicosities); chronic venous insufficiency; history of cancer; history of deep vein thrombosis (DVT) or pulmonary embolism (PE); history of heart failure (NYHA class III or IV); thrombophilia (hereditary or acquired); recent major surgery (6-12 weeks); recent serious trauma (6-12 weeks); hormone replacement therapy; advanced age greater than or equal to 75 years; morbid obesity (body mass index (BMI) 35 kg/m² or higher); and acute infectious disease contributing to hospitalization.

Treatment Group

The study was conducted in a prospective, randomized, double-blind, double dummy, active comparator-controlled, multicenter and multinational design. There were 8101 subjects were randomized at 556 study centers in 52 countries to one of the two treatment groups. Of the 8101 randomized subjects, 4050 were randomized to active rivaroxaban and 4051 were randomized to active enoxaparin.

Criteria for Evaluation

Efficacy had Primary and Secondary Endpoints.

Primary Endpoints: The primary efficacy variable was a composite of the number of events of: asymptomatic proximal DVT in lower extremity detected by mandatory bilateral lower extremity venous ultrasonography; symptomatic DVT in lower extremity, proximal or distal; symptomatic, non-fatal PE; and VTE-related death defined as either a well-documented fatal PE, or sudden death with no other plausible explanation. The two primary efficacy endpoints were (i) test of superiority at Day 35 (Days 29-41, inclusive) and (ii) test of non-inferiority at Day 10 (Days 6-15, inclusive).

Secondary Endpoints: The first major secondary efficacy endpoint was defined as a composite of the test of superiority at Day 35 with component of VTE-related death substituted by all-cause mortality, up to Day 35. The second major efficacy secondary efficacy endpoint was the test for superiority at Day 10 using the same composite endpoint as the Day 10 non-inferiority test.

The safety component of Criteria for Evaluation was the primary safety endpoint of incidence of the composite of treatment-emergent major bleeding events and non-major clinically relevant bleeding events observed during treatment and not later than 2 days after the last intake of double-blind study drug.

Health Economics and Outcomes

An additional study endpoint was health care resource utilization, as assessed by reason and duration of hospitalization, any visit to health care professionals not required by the protocol, any re-hospitalization and/or emergency room visit during the entire study period, any outpatient surgery/consultation not required by the protocol, and rehabilitation center stays following hospital discharge.

The final efficacy endpoint related to quality of life and patient reported outcomes, collected using the 12-item Short Form Health Survey (SF-12), Version 2.0, and the European Quality of Life five dimensions questionnaire (EQ-5D).

Pharmacokinetics

A subset of approximately 125 subjects was to be stratified into 5 groups (of 25 subjects) based on the major diagnoses. The following pharmacokinetic (PK) parameters were calculated on Day 1 and Day 10: area under the plasma concentration curve (AUC), area under the curve normalized to body weight (AUCₙₒᵣₘ), area under the curve for the expected dosing interval obtained after single dose administration (AUC_{τ}), AUC_{τ, norm}, maximum serum concentration (Cₘₐₓ), maximum and normalized serum concentration (C_{max, norm}), time to reach maximum concentration in plasma (tₘₐₓ), half-life (t_{1/2}), and total body clearance of drug from plasma calculated after oral administration (CL/F).

Pharmacodynamics

Full profile pharmacodynamics (PD) sampling was performed in parallel to the PK samples on Day 1 and Day 10. In addition, sparse PD sampling was performed in nearly all subjects at 5 predefined time points: prior to study drug administration, around the time of peak plasma concentrations (2 to 4 hours after intake of the rivaroxaban tablet or rivaroxaban placebo tablet) on the first, 10th and 35th day of study drug administration, and before study drug administration on Day 10.

Coagulation parameters assessed included international normalized ratio (INR), prothrombin time (PT), prothrombinase-induced clotting time (PiCT), and the fibrin degradation product, D-dimer. Pharmacodynamic parameters assessed included changes to baseline of the maximal effect (Eₘₐₓ) and changes to baseline at trough (E_{trough}).

Statistical Methods

All statistical tests were formally performed at a one-sided level of 0.025. However, following general conventions, p-values for superiority testing are presented as two-sided and p-values for non-inferiority testing are presented as one-sided.

For primary endpoints the multiple testing approach according to Hochberg was employed. If both primary efficacy endpoints were significant at the one-sided level of 2.5%, both primary efficacy endpoints were declared statistically significant. Otherwise, if the p-value for one of the primary endpoints was larger than 2.5% (one-sided), the p-value for the other primary endpoint must have been below 1.25% (one-sided) in order to declare statistical significance (for this primary endpoint alone).

For both primary efficacy endpoints, the relative risk ratio (rivaroxaban/enoxaparin) with respect to the incidences of the primary efficacy endpoint was estimated based on a stratified estimator using Mantel-Haenszel weights (based on sample sizes by geographic region). The corresponding asymptotic 2-sided 95% confidence interval based on the approximation to the normal distribution was determined. Sensitivity analyses were performed for the primary efficacy endpoints.

The primary safety outcome of the study was the treatment-emergent clinically relevant bleeding events, which were a composite of treatment-emergent adjudicated major bleeding events and clinically relevant non-major bleeding events.

Study Subjects:

There were generally no differences between the two treatment groups with respect to subject disposition, demographics, and baseline characteristics. Subjects in both treatment groups had a similar medical history. The mean number of days of hospitalization prior to treatment was 1.5 days (median 1.0 days). Median post randomization hospitalization time was 11 days, and median post randomization complete immobilization time was 4 days. The most common medical condition for hospitalization was acute infectious and inflammatory diseases including acute infectious diseases (47.3%), followed by heart failure (32.4%) and acute respiratory insufficiency (28.0%) (safety analysis set). Overall in the safety analysis set, the mean age was 69 years, and mean body mass index (BMI) was 28.2 kg/m². Sixty-nine percent of subjects were White, 20% were Asian, and 2% were Black. Of note, there were statistically significantly more males in the rivaroxaban group than in the enoxaparin group in all analysis sets (p=0.0061 safety set).

6005 subjects completed the full 35-day treatment period, 2958 (73.0%) in the active rivaroxaban group and 3047 (75.2%) in the active enoxaparin group. Mean duration of treatment for the entire treatment period was 32.4 days for active rivaroxaban and 8.6 days for active enoxaparin in the efficacy modified intent-to-treat (mITT) Day 35 analysis set. Mean compliance over the entire treatment period was 98.6% for rivaroxaban and 99.4% for enoxaparin (mITT Day 35). The mean time from randomization to the Day 10 visit ultrasonogram was 8.8 days (median 8.0 days) in both treatment groups, and to the Day 35 visit ultrasonogram was 35.2 days for rivaroxaban and 35.1 days for enoxaparin (median time 35 days in both treatment groups).

Efficacy Evaluation

Rivaroxaban 10 mg OD for a duration of 35 days was shown to be effective for the prevention of VTE in subjects with acute medical illnesses at risk for VTE. The primary efficacy variable was a composite of the number of events of (1) asymptomatic proximal DVT in lower extremity detected by mandatory bilateral lower extremity venous ultrasonography; (2) symptomatic DVT in lower extremity, proximal or distal; (3) symptomatic, nonfatal PE; and (4) VTE-related death. Efficacy was analyzed during the following phases of the study:
Rivaroxaban-enoxaparin phase (Day 1 to Day 10): Each treatment group received both active treatment and placebo; Day 1 to Day 10 (Day 6 to Day 15, inclusive used for efficacy data analysis).
Rivaroxaban-placebo phase (Day 10 to Day 35): Rivaroxaban subjects continued to receive active treatment; enoxaparin subjects continued to receive placebo; Day 10 to Day 35 (Day 16 to Day 41, inclusive used for efficacy data analysis).
Rivaroxaban-enoxaparin/placebo phase (Day 1 to Day 35): Rivaroxaban subjects received active treatment (35 days total), while enoxaparin subjects received active treatment for the first 10 days only, and then placebo for the remaining 25 days; Day 1 to Day 35; (Day 1 to Day 41 inclusive used for efficacy data analysis).
Follow-up phase: Subjects did not receive study drug. Started the day after the last intake of study drug, regardless of the duration of study drug administration, and ended on Day 90; (Last intake of study drug to Day 97 inclusive used for efficacy data analysis).
Observational phase: The entire treatment period and the subsequent follow-up period Randomization until Day 90 Visit (Day 1 to Day 97 inclusive used for efficacy data analysis).

The primary efficacy evaluation of the composite primary efficacy variable was performed at Day 35 and Day 10 endpoints.

At Day 35, the end of the rivaroxaban-enoxaparin/placebo phase (Day 1 to Day 35) (treatment phase), rivaroxaban (10 mg OD for 35 days) was statistically significantly superior to enoxaparin (40 mg sc for 10 days) in the mITT Day 35 analysis set (p=0.0211). The relative risk was 0.771 with the confidence interval (CI) ranging from 0.618 to 0.962. The relative risk reduction was 22.89%, (95% CI: 3.83% to 38.17%). The incidence of the primary efficacy composite outcome events at Day 35 was higher in the enoxaparin group (5.7% [175/3057]) than in the rivaroxaban group (4.4% [131/2967]) (mITT Day 35 analysis set).

At Day 10, the end of the rivaroxaban-enoxaparin phase (Day 1 to Day 10) (active control treatment phase), rivaroxaban 10 mg OD was statistically significantly non-inferior (with a relative margin of 1.5, one-sided p=0.0025) to enoxaparin 40 mg sc in the per protocol (PP) Day 10 analysis set. The relative risk was 0.968 with 95% CI ranging from 0.713 to 1.314. The relative risk reduction was 3.25% (95% CI −31.38% to 28.75%). The incidence of the primary efficacy composite outcome events at Day 10 was identical in both treatment groups in the PP Day 10 analysis set (rivaroxaban, 2.7% [78/2938]); enoxaparin, 2.7% [82/2993]).

In addition, the incidence of each of the individual components of the composite primary efficacy endpoint at Day 35 was lower in the rivaroxaban group than in the enoxaparin group in the mITT Day 35 analysis set. The enoxaparin group had a numerically higher incidence of VTE-related death (1.0%) than the rivaroxaban group (0.6%) at Day 35. At Day 35, the majority of events in both treatment groups were in the category asymptomatic proximal DVTs in lower extremity (rivaroxaban, 3.5%; enoxaparin, 4.4%).

The incidence of each of the individual components of the composite primary efficacy endpoint at Day 10 in the rivaroxaban group was similar to the enoxaparin group in the PP Day 10 analysis set.

| | Treatment group | |
|---|---|---|
| Primary endpoint/components | Rivaroxaban | Enoxaparin |
| Day 35 + 6: Modtfied intent-to-treat Day 35 | N = 2967 (100%) | N = 357 (100%) |
| Any event | 131 (4.4%) | 175 (5.7%) |
| Symptomatic non-fatal pulmonary embolism | 10 (0.3%) | 14 (0.5%) |
| Symptomatic deep vein thrombosis in lower extremity | 13 (0.4%) | 15 (0.5%) |
| Asymptomatic proximal deep vein thrombosis in lower extremity | 103 (3.5%) | 133 (4.4%) |
| Venous thromboembolism related death | 19 (0.6%) | 30 (1.0%) |
| Death (pulmonary embolism) | 1 (<0.1%) | 1 (<0.1%) |
| Death (pulmonary embolism cannot be excluded) | 18 (0.6%) | 29 (0.9%) |
| Day 10 + 5: Per protocol Day 10 | N = 2938 (100%) | N = 2993 (100%) |
| Any event | 78 (2.7%) | 82 (2.7%) |
| Symptomatic non-fatal pulmonary embolism | 86 (0.2%) | 2 (<0.1%) |
| Symptomatic deep vein thrombosis in lower extremity | 7 (0.2%) | 6 (0.2%) |
| Asymptomatic proximal deep vein thrombosis in lower extremity | 71 (2.4%) | 71 (2.4%) |
| Venous thromboembolism related death | 3 (0.1%) | 6 (0.2%) |
| Death (pulmonary embolism cannot be excluded) | 3 (0.1%) | 6 (0.2%) |

In the rivaroxaban-placebo phase (Day 10 to Day 35) (placebo control treatment phase), evaluated in a sensitivity analysis, rivaroxaban was statistically significantly superior to enoxaparin (p=0.0035) at Day 35 (mITT [Day 10 to Day 35] analysis set). The relative risk was 0.649, with the 95% confidence interval (CI) ranging from 0.485 to 0.867. The relative risk reduction was 35.14%, (95% CI: 13.27% to 51.50%). The incidence of the primary efficacy composite outcome events at Day 35 for the mITT (Day 10 to Day 35) analysis set was higher in the enoxaparin group (3.8%) than in the rivaroxaban group (2.5%). The enoxaparin group had a numerically higher incidence of VTE-related death (0.5%) than the rivaroxaban group (0.3%). The majority of events in both treatment groups were in the category asymptomatic lower proximal DVTs (rivaroxaban, 2.1%; enoxaparin, 2.9%).

| Events (Day 10 + 5 up to Day 35 + 6 days) | Treatment group | |
|---|---|---|
| Primary endpoint/components | Rivaroxaban | Enoxaparin |
| Modified intent-to-treat (Day 10 to Day 35) analysis set | N = 2934 (100%) | N = 3017 (100%) |
| Any event | 72 (2.5%) | 114 (3.8%) |
| Symptomatic non-fatal pulmonary embolism | 1 (<0.1%) | 10 (0.3%) |
| Symptomatic deep vein thrombosis in lower extremity | 3 (0.1%) | 7 (0.2%) |
| Asymptomatic proximal deep vein thrombosis in lower extremity | 81 (2.1%) | 89 (2.9%) |
| Venous thromboembolism related death | 9 (0.3%) | 15 (0.5%) |
| Death (pulmonary embolism) | 1 (<0.1%) | 0 |
| Death (pulmonary embolism cannot be excluded) | 8 (0.3%) | 15 (0.5%) |

Additional sensitivity analyses confirmed the results of the primary efficacy analyses.

Subgroup analyses: At Day 35 rivaroxaban was generally more effective in reducing the incidence of VTE compared with enoxaparin across a diverse range of primary medical illnesses, other risk factors, demographic factors, and geographic regions. Of note:
Rivaroxaban appeared to be more effective in subjects in Western Europe (including Australia, New Zealand, and Israel) than in other regions compared with enoxaparin, with a relative risk of 0.5, 95% CI 0.319 to 0.783; (mITT Day 35 analysis set)
Rivaroxaban appeared to be more effective than enoxaparin in the age subgroup 75 years compared with enoxaparin (relative risk 0.624; 95% CI 0.458 to 0.848; mITT Day 35 analysis set) than in other age subgroups
Rivaroxaban appeared to be more effective in subjects with acute infectious and inflammatory disease (relative risk 0.657; 95% CI 0.475 to 0.909; mITT Day 35 analysis set) than other medical conditions.

An exception was that rivaroxaban appeared to be less effective in subjects with active cancer (relative risk 1.340; 95% CI 0.706 to 2.542; mITT Day 35 analysis set), compared to enoxaparin.

With few exceptions, at Day 10 rivaroxaban was generally as effective as enoxaparin in reducing the incidence of VTE across a diverse range of primary medical illnesses, other risk factors, demographic factors, and geographic regions. Some exceptions were:
Rivaroxaban appeared to be less effective than enoxaparin for the age subgroup 65 to <75 years (relative risk=1.88; 95% CI 1.017 to 3.461; PP Day 10 analysis set) than other age subgroups.
Rivaroxaban appeared to be less effective in subjects with duration of complete immobilization post randomization of 4-6 days (relative risk=1.75; 95% CI 0.914 to 3.363; PP Day 10 analysis set) compared with enoxaparin.
Rivaroxaban appeared to be more effective in subjects with duration of complete immobilization >6 days post randomization (relative risk=0.671; 95% CI 0.369, to 1.222; PP Day 10 analysis set), compared with enoxaparin.
Rivaroxaban appeared to be less effective in subjects with active cancer (relative risk 2.500; 95% CI 0.797 to 7.847; PP Day 10 analysis set) compared with enoxaparin.

An evaluation of secondary efficacy endpoints led to the following results:

When the component of VTE-related death (in the primary variable) was substituted by all-cause mortality, rivaroxaban appeared to be more effective than enoxaparin, but statistically significant superiority of rivaroxaban over enoxaparin in the mITT Day 35 analysis set (expanded to include all subjects with cause of death not VTE related) was not demonstrated. In the rivaroxaban-enoxaparin/placebo phase (Day 1 to Day 35), the incidence of the composite endpoint, including all-cause mortality at Day 35 was 8.6% (266/3096) in the rivaroxaban group and 9.2% (293/3169) in the enoxaparin group, and the relative risk was 0.931, with the 95% CI ranging from 0.795 to 1.091. The second major efficacy endpoint (superiority at Day 10 using the same composite endpoint as the Day 10 non-inferiority test) was also not met.

For the incidence of symptomatic VTE, excluding VTE-related death, numerical results favored rivaroxaban at Day 35 (rivaroxaban, 0.6%; enoxaparin, 0.7%) and Day 90 (rivaroxaban, 0.7%; enoxaparin, 0.9%), and enoxaparin at Day 10 (rivaroxaban, 0.5%; enoxaparin, 0.3%); there was no statistical significance found. A similar pattern was found for symptomatic VTE including death: Day 35 (rivaroxaban, 1.0%; enoxaparin, 1.4%) and Day 90 (rivaroxaban, 1.7%; enoxaparin, 1.9%), and Day 10 (rivaroxaban, 0.7%; enoxaparin, 0.6%).

The net clinical benefit, which was defined as a composite of the primary efficacy endpoint plus major and clinically relevant non-major bleeding events, rivaroxaban had a less favorable profile, compared with enoxaparin, mainly due to the higher number of subjects with bleeding events in the rivaroxaban group: net clinical benefit at Day 35 (rivaroxaban, 9.4%; enoxaparin, 7.8%; mITT Day 35, statistically significant in favor of enoxaparin [two-sided p-value 0.0224]) and at Day 10 (rivaroxaban, 4.5%; enoxaparin, 3.9%; PP Day 10).

| Net clinical benefit | Treatment group | |
|---|---|---|
| endpoint/components | Rivaroxaban | Enoxaparin |
| Day 35 + 6: Modified intent-to-treat Day 35 plus major and clinically | N = 3042 (100%) | N = 3082 (100%) |
| relevant bleeds (primary analysis set) | | |
| Any event | 286 (9.4%) | 240 (7.8%) |
| Symptomatic non-fatal pulmonany embolism | 10 (0.3%) | 14 (0.5%) |
| Symptomatic deep vein thrombosis in lower extremity | 13 (0.4%) | 15 (0.5%) |
| Major bleeding | 43 (1.4%) | 15 (0.5%) |
| Clinically relevant non-major bleeding | 124 (4.1%) | 52 (1.7%) |
| Asymptomatic lower proximal deep vein thrombosis | 104 (3.4%) | 133 (4.3%) |
| Venous thromboembolism related death | 19 (0.6%) | 30 (1.0%) |
| Death (pulmonary embolism) | 1 (<0.1%) | 1 (<0.1%) |
| Death (pulmonary embolism cannot be excluded) | 18 (0.6%) | 29 (0.9%) |
| Day 10 + 5: Per protocol Day 10 plus major and clinically relevant | N = 2950 (100%) | N = 3007 (100%) |
| bleeds (primary analysis set)^{a} | | |
| Any event | 134 (4.5%) | 118 (3.9%) |
| Symptomatic non-fatal pulmonary embolism | 6 (0.2%) | 2 (<0.1%) |
| Symptomatic deep vein thrombosis in lower extremity | 7 (0.2%) | 6 (0.2%) |
| Major bleeding | 9 (0.3%) | 4 (0.1%) |
| Clinically relevant non-major bleeding | 51 (1.7%) | 32 (1.1%) |
| Asymptomatic lower proximal deep vein thrombosis | 71 (2.4%) | 71 (2.4%) |
| Venous thromboembolism related death | 3 (0.1%) | 6 (0.2%) |
| Death (pulmonary embolism) | 3 (0.1%) | 6 (0.2%) |

The incidence of major vascular events (cardiovascular [CV] death, acute myocardial infarction, or acute ischemic stroke) was the same in both treatment groups at Day 10 (1.0%) and at Day 90 (2.8%), and slightly higher in the rivaroxaban group at Day 35 (rivaroxaban, 1.8%; enoxaparin, 1.6%).

The incidence of all-cause mortality at Day 90 was slightly higher in the rivaroxaban group (rivaroxaban, 6.7%; enoxaparin, 6.2%), but no statistically significant difference between treatment groups was seen (p-value 0.400).

Health economics and outcomes analyses did not reveal any notable differences between the treatment groups, but confirmed the fact that these patients are severely ill, with a duration of hospitalization of 11 days and an average of 4 days of complete immobilization.

Pharmacokinetic Evaluation

No evidence of relevant drug accumulation beyond steady state was observed between Day 1 and Day 10 in all subject groups, although drug exposure at Day 1 (overall, AUC 1642 ug*h/L; Cmax 176 ug/L) tended to be higher than at Day 10 (overall, AUC 1355 ug*h/L; Cmax 189 ug/L) for all subject subgroups.

Drug exposure was comparable between subject groups. No relevant differences in AUC and Cmax were observed, with subjects with acute infectious disease at the lower end (Day 10: AUC 1192 ug*h/L; Cmax 178.9 ug/L) and subjects with congestive heart failure (CHF) NYHA IV at the upper end (Day 10: AUC 1436 ug*h/L; Cmax 191.8 ug/L) of the overall subject data. The tendency to higher AUC (i.e., lower clearance) data, especially for some subject subgroups (such as CHF and acute respiratory insufficiency), may be explained by the varying severity of the underlying disease.

Drug exposure at steady state and CL/F for this study population in general were in the range of results previously obtained with 10 mg rivaroxaban in both healthy adults (phase 1 data pool; age ranging from 18 to 83 years) as well as in VTE-major orthopedic surgery (MOS) subject populations.

Rivaroxaban plasma concentration/PT response relationship was comparable between subject groups and again similar to data reported for VTE-MOS subjects.

Pharmacodynamic Evaluation

Two sets of samples were evaluated for PD parameters: Full profile PD sampling in parallel to the PK samples on Day 1 and Day 10. In addition, sparse PD sampling in nearly all subjects at 5 predefined time points: prior to study drug administration, approximately the time of peak plasma concentrations (2 to 4 hours after intake of the rivaroxaban tablet or rivaroxaban placebo tablet) on the first, 10th and 35th day of study drug administration, and before study drug administration on Day 10.

Prothrombin times from full profile sampling following administration of 10 mg rivaroxaban were comparable between the subgroups (overall median values, peak Day 1, 23.5 sec; peak Day 10, 23.2 sec).

The PT data from sparse and full sampling schemes are comparable for medically ill subjects and also do not differ from data obtained from previous studies in other study populations (subjects with acute decompensated heart failure, chronic stable heart failure, orthopedic surgery patients).

Enoxaparin has no influence on PT, as expected from previous investigations. Prothrombin times from sparse data sampling were: Baseline, 14.4 sec; Day 1 peak, 14.8 sec; Day 10 peak, 14.2 sec, Day 35 peak, 13.8 sec, median values). Corresponding rivaroxaban median values: Baseline, 14.3 sec; Day 1 peak, 19.3 sec; Day 10 peak, 19.3 sec, Day 35 peak, 18.6 sec.

An overall decrease in D-dimer following administration of rivaroxaban over the course of the treatment was observed in the entire study population (rivaroxaban median values: Baseline, 0.94 μg/mL; Day 1 peak, 0.90 μg/mL; Day 10 peak, 0.64 μg/m L; Day 35 peak, 0.45 μg/mL; enoxaparin median values: baseline, 0.95 μg/mL; Day 1 peak, 0.94 μg/mL; Day 10 peak, 0.66 μg/mL; Day 35 peak, 0.67 μg/m L).

Subgroup analyses from subjects classified as NYHA III and IV with full PD sampling were consistent with the overall results. In subjects with acute infectious disease or acute respiratory insufficiency, D-dimer appears to be unaffected by rivaroxaban (acute infectious disease, median values: Baseline, 1.76 μg/mL; Day 1 peak, 2.07 μg/mL; Day 10 trough, 1.74 μg/mL; Day 10 peak, 2.13 μg/mL; acute respiratory insufficiency, median values: baseline, 1.48 μg/mL; Day 1 peak, 1.44 μg/mL; Day 10 trough, 1.18 μg/mL; Day 10 peak, 1.37 μg/mL). This is probably due to the low number of rivaroxaban subjects and high variability of the test. D-dimers appear also to be unaffected by enoxaparin in subjects with acute respiratory insufficiency (median values: Baseline, 0.57 μg/mL; Day 1 peak, 0.66 μg/mL; Day 10 trough, 0.46 μg/mL; Day 10 peak, 0.70 μg/mL).

The prothrombinase induced clotting time (PiCT) (one step) data from sparse sampling were analyzed in both rivaroxaban and enoxaparin-treated subjects. Rivaroxaban prolonged the PiCT one step assay by 14.6 seconds at Day 1, and by 13.1 seconds on Day 10. No change in the clotting time was observed for enoxaparin-treated subjects. Expressed in units of μg/L, mean peak concentration of rivaroxaban were 198.8 μg/L on Day 1, 202.78 μg/L on Day 10 and 167.85 μg/mL on Day 35.

The PiCT (two step) data from sparse sampling were analyzed in both rivaroxaban and enoxaparin-treated subjects. Rivaroxaban had little effect on the PiCT two step assay and no difference was observed between baseline and peak measurements on Day 1 and between trough and peak values on Day 10. Enoxaparin prolonged the PiCT assay by 23.6 seconds on Day 1 and by 19.8 seconds on Day 10. No change in the clotting time was observed for rivaroxaban-treated subjects. Expressed in units of IU/mL, mean peak concentration of enoxaparin was 0.32 IU/mL on Day 1 and 0.31 IU/mL on Day 10. The baseline value was below the lower limit of quantitation on Day 1 and trough was reported to be 0.11 IU/mL po Day 10. For rivaroxaban-treated subjects, peak values were below the lower limit of quantitation on both Day 1 and Day 10.

Safety Evaluation

The safety analysis was performed on 7998 randomized subjects who received at least one dose of study drug, rivaroxaban (3997 subjects) or enoxaparin (4001 subjects).

The primary safety outcome of the study was the assessment of clinically relevant bleeding events, which are a composite of treatment-emergent adjudicated major bleeding events and clinically relevant non-major bleeding events.

Overall, the incidence of all adjudicated bleeding events (major, clinically relevant non-major, and minimal) was higher in the rivaroxaban group when compared with the enoxaparin group in all phases of the study: the rivaroxaban-enoxaparin/placebo phase (Day 1 to 35), the rivaroxaban-enoxaparin phase (Day 1 to 10), and the rivaroxaban-placebo phase (Day 10 to 35). In the follow-up phase of the study (Day 35 to 90), the bleeding events were comparable in the two treatment groups.

The incidence of clinically relevant bleeding events in the rivaroxaban and enoxaparin groups was reported in 164 (4.1%) and 67 (1.7%) subjects in the rivaroxaban-enoxaparin/placebo treatment phase (relative risk of 2.455, <0.0001), 111 (2.8%) and 49 (1.2%) subjects in the rivaroxaban-enoxaparin treatment phase (relative risk of 2.272, <0.0001), and 56 (1.4%) and 19 (0.5%) subjects in the rivaroxaban-placebo treatment phase (relative risk 2.958, <0.001), respectively. The confidence intervals and p-values for the weighted relative risks of bleeding events in the three treatment phases were of significance in favor of enoxaparin.

Major bleeding events were reported in 64 (1.6%) subjects in the rivaroxaban group (74 major bleeding events) and 37 (0.9%) subjects in the enoxaparin group (42 major bleeding events). By phase, major bleeding events in the rivaroxaban-enoxaparin/placebo phase (Day 1 to Day 35) were reported in 43 (1.1%) vs 15 (0.4%) subjects, in the rivaroxaban-enoxaparin phase (Day 1 to Day 10) in 24 (0.6%) vs 11 (0.3%) subjects, in the rivaroxaban-placebo-phase (Day 10 to Day 35) in 19 (0.5%) and 4 (<0.1%) subjects in the rivaroxaban and enoxaparin groups, respectively. In the follow-up phase the incidence was the same in either treatment group (23 [0.6%] subjects)
A total of 8 (0.1%) subjects were reported with treatment-emergent (Day 1 to 35) fatal bleeding events leading to death, 7 in the rivaroxaban group (3 due to pulmonary site bleeding, 2 due to intracranial bleeding events, and 1 each due to retroperitoneal and gastrointestinal bleeding event) and 1 in the enoxaparin group (due to tracheal bleeding event). Majority of the fatal bleeding events occurred during the rivaroxaban-enoxaparin phase (Day 1 to 10) with 5 cases in the rivaroxaban group and 1 in the enoxaparin group.
In the rivaroxaban-enoxaparin/placebo phase (Day 1 to Day 35), the most prominent difference in the number of subjects with bleeding events was reported (rivaroxaban vs enoxaparin) for the intracranial bleeding site (4 [0.1%] vs 2 [<0.1%] subjects), retroperitoneal bleeding site (3 [<0.1%] vs 0 subjects), and pulmonary bleeding site (3 [<0.1%] vs 0 subjects).
The number of subjects with critical site bleeding events was also higher in the rivaroxaban group when compared with enoxaparin. In the rivaroxaban-enoxaparin/placebo phase (Day 1 to Day 35), the most prominent difference in the incidence of critical site bleeding events was reported (rivaroxaban vs enoxaparin) for the intracranial bleeding site (4 [0.1%] vs 2 [<0.1%] subjects), retroperitoneal bleeding site (3 [<0.1%] vs 0 subjects), and pulmonary bleeding site (3 [<0.1%] vs 0 subjects).
Bleeding events that resulted in a fall of Hb to ≥2 g/dL (0.8% vs 0.2%) or that required blood transfusions of 2 units (0.6% vs 0.2%) were also higher in the rivaroxaban group when compared with the enoxaparin group.

The number of subjects with multiple bleeding events was slightly higher in the rivaroxaban group in comparison to enoxaparin.

The number of subjects with clinically relevant non-major bleeding events was higher in rivaroxaban (158 [4.0%] subjects with 184 bleeding events) in comparison to enoxaparin (83 [2.1%] subjects with 90 bleeding events). However, the incidence of minimal bleeding events was comparable in the two treatment groups with reports of 382 (9.6%) subjects in the rivaroxaban group and 330 (8.2%) subjects in the enoxaparin group.

The incidences of adjudicated treatment-emergent bleeding events analyzed in the four geographic regions (Western-Europe, Eastern-Europe, Asia/Africa, and America) did not show any consistent subgroup findings. Analysis of treatment-emergent bleeding events was also performed for numerous specified covariates grouped into demography, immobilization, active and acute illness (such as cancer), history of disease (including cancer, infections, DVT or PE, obesity, heart failure, etc.), and inhibitors and inducers of the cytochrome P450 enzyme, CYP3A4. Descriptive values consistently favored enoxaparin over rivaroxaban.

The number of subjects with investigator-reported bleeding events was consistent with the adjudicated bleeding events showing a higher incidence in the rivaroxaban group in comparison to enoxaparin. The incidences of the adverse events (AEs) without bleeding and the AEs with bleeding were similar.

In the rivaroxaban-enoxaparin/placebo (treatment-emergent) phase (Day 1 to Day 35), the investigators reported an incidence of 12.5% treatment-emergent adverse events (TEAEs), 6.9% drug-related TEAEs, and 2.0% serious adverse events (SAEs) in the rivaroxaban group and an incidence of 8.5% TEAEs, 4.2% drug-related TEAEs, and 0.8% SAEs in the enoxaparin group. The incidence of investigator-reported TEAEs leading to permanent discontinuation of the study drug was 3.1% (rivaroxaban) and 1.5% (enoxaparin).

The incidence of investigator-reported deaths in the entire study duration (Day 1 to 90) was comparable in both treatment groups with 280 (7.0%) deaths in the rivaroxaban group and 262 (6.5%) deaths in the enoxaparin group. The incidence of VTE-related deaths was 39 (1.0%) subjects in the rivaroxaban group and 48 (1.2%) subjects in the enoxaparin group. Pulmonary embolism was confirmed in 3 subjects (2 rivaroxaban and 1 enoxaparin).

The incidence of hepatic disorders was lower in the rivaroxaban group compared to enoxaparin. The liver-related laboratory test abnormalities showed notable elevations in the hepatic enzymes and TBL in the enoxaparin group during the treatment-emergent phase.

The adjudicated CV events were comparable in the two treatment groups, both in the treatment-emergent and the follow-up phase of the study.

The changes in laboratory parameters including amylase, lipase, and platelet number were similar in both treatment groups. There were no notable findings in any of the clinical laboratory investigations (other than the hepatic enzyme elevations), or other safety evaluations such as vitals or electrocardiograms (ECGs).

Overall Conclusion for Study 1: MAGELLAN

The MAGELLAN study (Study 1) met its protocol specified primary efficacy outcomes for rivaroxaban versus enoxaparin/placebo at both Day 10 and Day 35. Bleeding rates in MAGELLAN were low overall and consistent with rates in prior trials. The enoxaparin bleeding rate in MAGELLAN is comparable to that reported in prior trials in this indication. Nonetheless, the incidences of clinically relevant bleeding events including both the component of major bleeding events as well as the component of non-major clinically relevant bleeding events were increased in rivaroxaban treated subjects compared to enoxaparin followed by placebo treated subjects in a statistically significant manner during all three specified study phases. The non-bleeding safety of rivaroxaban in this patient population was in line with previous clinical experience, and not different from enoxaparin followed by placebo. There was no imbalance of drug-related treatment-emergent AE, SAE, CV events, or hepatic diseases evaluated using the Standard Medical Dictionary for Regulatory Activities (MedDRA) Queries (SMQs), or liver function tests (LFTs).

Study 2: Mariner

The primary objective of Study 2 (MARINER) was to assess the efficacy and safety of rivaroxaban, compared with placebo in the prevention of symptomatic venous thromboembolism (VTE; lower extremity deep vein thrombosis [DVT] and non-fatal pulmonary embolism [PE]) and VTE-related death (death due to PE or death in which PE could not be ruled out as the cause) post-hospital discharge in high-risk, medically ill subjects.

The secondary objectives were to compare rivaroxaban with placebo in the following post-hospital discharge outcomes in high-risk, medically ill subjects:
VTE-related death.
Symptomatic VTE.
The composite of symptomatic VTE and all-cause mortality (ACM).
The composite of symptomatic VTE, myocardial infarction (MI), non-hemorrhagic stroke and cardiovascular (CV) death (death due to a known CV cause and death in which a CV cause could not be ruled out; by this definition, a VTE-related death was considered a CV death).
ACM.

The exploratory objectives were to compare rivaroxaban with placebo, in the following post-hospital discharge outcomes in high-risk, medically ill subjects:
Symptomatic lower extremity DVT.
Symptomatic non-fatal PE.
Symptomatic upper extremity DVT.
MI.
Non-hemorrhagic stroke.
Re-hospitalization for symptomatic VTE within 30 days after randomization.

The safety objectives were to compare rivaroxaban with placebo in the following bleeding outcomes in high-risk, medically ill subjects:
Major bleeding using validated International Society on Thrombosis and Haemostasis (ISTH) bleeding criteria.
Non-major clinically relevant (NMCR) bleeding.
Other bleeding.

Overall safety was also assessed.

Methodology

There were 7 global amendments to the original protocol, the methodology described below reflect those in the final protocol amendment (INT-7).

This was a multicenter, prospective, randomized, double-blind, placebo-controlled, event-driven study conducted in multiple geographic regions (North America, South America, Eastern Europe, Western Europe, and Other locations) that evaluated rivaroxaban, compared with placebo, in the prevention of symptomatic VTE events and VTE-related deaths for a period of 45 days post-hospital discharge in high-risk, medically ill subjects. The study consisted of a screening phase, a 45-day double-blind treatment phase, and a 30-day safety follow-up period (which was also the end of study [EOS]). The total duration for a subject who completed the study (including the safety follow-up period) after randomization was 75 (±5) days.

The index hospitalization was at least 3 and no more than 10 consecutive days in duration. Subjects had other risk factors for VTE, as demonstrated by a total modified International Medical Prevention Registry on Venous Thromboembolism (IMPROVE) VTE risk score of or VTE risk score of 2 or 3 with D-dimer >2 times upper limit of normal (xULN). Subjects received thromboprophylaxis during the index hospitalization with low molecular weight heparin (LMWH) or unfractionated heparin (UFH). Any subject with a medical condition that required use of any parenteral or oral anticoagulation during the study or who had a particularly increased risk of bleeding or who used medications that could have interacted with the study drug was excluded from participating.

Randomization occurred on the same day or the day after the subject left the hospital, and occurred at the hospital, clinic, or other discharge destination. Eligible subjects were to be randomly assigned (1:1) by strata (by creatinine clearance [CrCl] level [≥30-<50 mL/min versus ≥50 mL/min] and by country) to receive either rivaroxaban or placebo. Subjects with a baseline CrCl of ≥50 mL/min were to receive rivaroxaban 10 mg once daily (QD) or placebo QD. A dose adjustment to rivaroxaban 7.5 mg QD was to be implemented for subjects with a baseline CrCl of ≥30 to <50 mL/min. Study drug started at randomization (Day 1) and continued until Day 45 (inclusive). The subject was assessed 30 days later for safety follow-up.

Number of Subjects (Planned and Analyzed)

Planned: In this event-driven study, the targeted total number of subjects with a primary efficacy outcome event was 161. To observe this targeted total number of subjects, it was originally estimated that approximately 8,000 subjects needed to be randomized to either rivaroxaban or placebo in a 1:1 ratio. This estimate was based on an estimated placebo incidence rate of the primary efficacy outcome of 2.5%. In the event that the actual observed blinded pooled event rate was lower than the assumed rate, more subjects were permitted to be enrolled to accumulate additional outcome events. The blinded pooled event rate, as actually observed during the study, was lower than the originally assumed rate; therefore, the initially planned maximum number of randomized subjects (9,000) was increased to approximately 12,000 subjects to accumulate additional outcome events. Randomization was stopped at 12,024 subjects.

Analyzed: In total, 13,708 subjects were screened for eligibility (including subjects prescreened for eligibility based on D-dimer results) at 713 study centers in 36 countries. A total of 1,685 subjects were considered screen failures, with the primary reasons being a D-dimer value ≤2×ULN (1,018 subjects) and ineligible for entry (513 subjects). A total of 12,024 subjects were randomized at 671 study centers in 36 countries to 1 of the 2 treatment groups. Of the 12,024 randomized subjects, 12,019 were included in the intention-to-treat (ITT) analysis (6,007 in the rivaroxaban group and 6,012 in the placebo group). The number of subjects screened and randomized, and the number of subjects included in each analysis set by treatment group are summarized below.

Data Set Analyzed: All Subjects Analysis Set

| | Rivaroxaban | Placebo | Total |
|---|---|---|---|
| | n (%) | n (%) | n (%) |
| Screened | NA | NA | 13708 |
| Randomized | 6007 (100) | 6017 (100) | 12024 (100) |
| ITT analysis set | 6007 (100) | 6012 (99.9) | 12019 (100) |
| Safety analysis set | 5982 (99.6) | 5980 (99.4) | 11962 (99.5) |

ITT = intention-to-treat;
NA = not applicable;
PP = per-protocol.
NOTE:
The ITT analysis set was to include all randomized subjects who had signed a valid informed consent. Five randomized subjects were excluded from the ITT analysis set. Two of these subjects did not have valid informed consent and 3 subjects (at 1 study center) were randomized before the study center received Health Authority approval.
NOTE:
The PP analysis set is a subset of the ITT analysis set, and included all subjects in the ITT analysis set except for those with predefined key protocol deviations (subset of major protocol. deviations).
NOTE:
The safety analysis set includes all subjects in the ITT analysis set who took at least 1 dose of study drug.
NOTE:
Percentages based on number or subjects randomized.

Diagnosis and Main Criteria for Inclusion:

The target population consisted of men and women aged 40 years who were hospitalized for at least 3 and no more than 10 consecutive days prior to randomization for a specific acute medical illness (i.e., heart failure [HF] with a reduced left ventricular ejection fraction [LVEF ≤45%], acute respiratory insufficiency or acute exacerbation of chronic obstructive pulmonary disease [COPD], acute ischemic stroke [including spinal cord infarction if no evidence of intramedullary, subdural or epidural hemorrhage], acute infectious disease, or inflammatory disease, including rheumatic disease). Subjects also had other risk factors for VTE, as demonstrated by a total modified IMPROVE VTE risk score of ≥4 or VTE risk score of a 2 or 3 with D-dimer >2×ULN. Subjects received thromboprophylaxis during the index hospitalization with LMWH or UFH.

Test Product, Dose and Mode of Administration, Batch No.: Rivaroxaban was supplied as 10 mg and 7.5 mg tablets for oral administration. The batch numbers of rivaroxaban were as follows: AS020, KM500B5, KM500WL, KM60030, KM60031, KM600FS, and KM600FV.

Reference Therapy, Dose and Mode of Administration, Batch No.: Matching placebo tablets were supplied for oral administration; there were no visible differences between the 2 rivaroxaban strengths and the matching placebo tablets. The batch numbers of placebo were: KM500B4, KM500BN, KM500WH, KM60028, KM60029, and KM600JX.

Duration of Treatment: The duration of treatment for randomized subjects was 45 days. The first dose was taken on the day of randomization (Day 1). Subjects were instructed to discontinue study drug after they took their dose on Day 45.

Criteria for Evaluation: This was a clinical outcomes study. The primary efficacy outcome was the composite of all symptomatic VTE events and VTE-related death from randomization up to Day 45. Secondary efficacy outcomes were: VTE-related death; symptomatic VTE; the composite of symptomatic VTE and ACM; the composite of symptomatic VTE, MI, non-hemorrhagic stroke and CV death; and ACM.

Safety assessment was based on bleeding events classified by validated ISTH criteria as major bleeding (principal safety outcome), NMCR bleeding, or Other bleeding. A major bleeding event by ISTH criteria was defined as overt bleeding associated with: a fall in hemoglobin of 2 g/dL or more or leading to a transfusion of 2 or more units of packed red blood cells or whole blood, or bleeding that occurred in a critical site (intracranial, intraspinal, intraocular, pericardial, intra-articular, intramuscular with compartment syndrome, retroperitoneal), or had a fatal outcome. The safety outcome of NMCR bleeding was defined as overt bleeding not meeting ISTH criteria for major bleeding, but associated with medical intervention, unscheduled contact (visit or telephone call) with a physician, (temporary) cessation of study treatment, or associated with discomfort for the subject such as pain or impairment of activities of daily life. Other bleeding was defined as any other overt bleeding episodes that did not meet ISTH criteria for major bleeding or NMCR bleeding. Overall safety was also assessed.

In addition, plasma concentrations were measured for pharmacokinetic (PK) evaluations in a subpopulation of approximately 300 subjects treated with rivaroxaban (˜100 subjects with baseline CrCl ≥30-<50 mL/min and ˜200 subjects with baseline CrCl ≥50 mL/min). Although samples were also collected from approximately 300 subjects treated with placebo, only samples taken from subjects receiving rivaroxaban were measured.

Statistical Methods

The primary and secondary efficacy outcomes were analyzed based on the time from randomization to the first occurrence in the ITT analysis set and Up-to-Day 45 analysis phase. Adjudicated results (by the independent blinded Clinical Event Committee [CEC]) were used for the analysis. A Cox proportional hazards model, stratified by subjects with CrCl ≥30 to <50 mL/min versus subjects with CrCl ≥50 mL/min, with the treatment (as randomized) as the only covariate was used for each analysis. A 2-sided p-value was reported, and if it was less than the 2-sided alpha of 0.05, then superiority of the study drug was to be declared when the observed survival functions favored rivaroxaban. The point estimate and corresponding 95% confidence interval (CI) for the hazard ratio (HR; rivaroxaban to placebo) were provided based on the Cox proportional hazards model. To control the family-wise type I error rate at alpha of 0.05 (2-sided) in testing for efficacy outcomes, if superiority of rivaroxaban over placebo for the primary efficacy outcome was established, superiority of rivaroxaban over placebo on secondary outcomes was tested sequentially using a closed testing procedure in a hierarchical order, each at alpha of 0.05 (2-sided).

The principal safety outcome was major bleeding based on validated ISTH criteria. Treatments were compared using the same statistical model (i.e., Cox proportional hazards model) as that for the primary efficacy outcome. The same analyses were used for NMCR bleeding. Safety analyses were based on the safety analysis set and on-treatment analysis phase. Bleeding events were analyzed based on time from randomization to the first occurrence.

The benefit-risk of rivaroxaban versus placebo was evaluated based on the excess number of events between treatments for events intended to be prevented (benefits) and events that were caused (risks).

Unless otherwise stated, analysis of all efficacy and safety outcome events was based on CEC adjudication.

Results

Study Population:

Of the 12,024 subjects randomized, 12,019 were included in the ITT analysis set (6,007 in the rivaroxaban group and 6,012 in the placebo group); 2 subjects were excluded for not having valid informed consent and 3 subjects for being randomized prior to Health Authority approval. A total of 11,962 subjects in the ITT analysis set took at least 1 dose of study drug and were included in the safety analysis set (5,982 rivaroxaban and 5,980 placebo subjects).

According to the investigator, 10,897 (91.1%) subjects in the safety analysis set completed study treatment (5,457 [91.2%] in the rivaroxaban group and 5,440 [91.0%] in the placebo group) and 1,065 (8.9%) subjects prematurely discontinued study drug (525 [8.8%] subjects in the rivaroxaban group and 540 [9.0%] in the placebo group). The most frequent reason for premature discontinuation of study drug was subject choice (325 [2.7%] subjects), followed by adverse event (AE) other than efficacy and bleeding events (311 [2.6%] subjects), and Other reasons (170 [1.4%] subjects).

For the purpose of statistical analysis, a subject was considered a completer of double-blind treatment if the subject's last dose of study drug was at least on Day 45, on the date of death, or on date of the primary efficacy outcome. Based on this definition, 10,943 (91.5%) subjects were considered completers of double-blind treatment (5,476 [91.5%] in the rivaroxaban group and 5,467 [91.4%] in the placebo group).

According to the investigator, 11,745 (97.7%) subjects in the ITT analysis set completed the study, 229 (1.9%) subjects died, and 45 (0.4%) subjects discontinued the study prematurely. The most frequent reason for discontinuation from the study was consent withdrawn by subject (40 [0.3%] subjects).

For the purpose of statistical analysis, a subject was considered a non-completer of the study if the subject's last outcome-evaluation date was before the Day 45 date and the subject did not have a primary efficacy outcome or death before Day 45. Based on this definition, 43 (0.4%) subjects were considered non-completers (19 [0.3%] subjects in the rivaroxaban group and 24 [0.4%] subjects in the placebo group).

In general, the demographic and baseline characteristics were well balanced between treatment groups. Across both treatment groups, a greater proportion of subjects were male (6,284 [52.3%] subjects), white (11,590 [96.4%] subjects), and not Hispanic or Latino (11,000 [91.5%]). The mean age was 69.7 years (median: 70.0 years; range: 40-98 years).

Of the 12,019 subjects in the ITT analysis set, 712 (5.9%) subjects had major protocol deviations. The distribution of major protocol deviations was similar in both treatment groups. The most frequently reported major protocol deviation was that a subject entered the study but did not satisfy entry criteria (612 [5.1%] subjects) followed by received a disallowed concomitant treatment (99 [0.8%] subjects).

A total of 11,390 subjects (5,711 rivaroxaban and 5,679 placebo subjects) met the per-protocol definition and were included in the per-protocol (PP) analysis set. Subjects with key protocol deviations (subset of major protocol deviations) were excluded from the PP analysis set.

According to the protocol, treatment duration was defined as 45 days. The mean duration of treatment exposure was 43.0 days (median: 45.0 days): 43.1 days in the rivaroxaban group and 42.9 days in the placebo group. Treatment compliance was similar in both treatment groups. Across both treatment groups compliance was >90% for 11,157 (93.3%) subjects.

Efficacy Results

The results of the primary and secondary efficacy outcome analyses are summarized in the table below:

Hazard Ratio and 95% Confidence Interval for Time to the First Occurrence of the Primary and Secondary Efficacy
Outcomes, Up-to-Day 45 as Adjudicated by the CEC (Study RIVAROXDVT3002: Intention-to-Treat Analysis Set)

| | Rivaroxaban | | Placebo | | Rivaroxaban vs Placebo | |
|---|---|---|---|---|---|---|
| | | Event Rate | | Event Rate | Hazard Ratio | |
| Outcomes | n/N (%) | (100 pt-45 dy) | n/N (%) | (100 pt-45 dy) | (95% CI) [1] | p-value [2] |
| Primary efficacy outcome | 50/6007 (0.83) | 0.84 | 66/6012 (1.10) | 1.11 | 0.76 (0.52, 1.09) | 0.136 |
| Symptomatic lower extremity DVT | 4/6007 (0.07) | 0.07 | 13/6012 (0.22) | 0.22 | 0.31 (0.10, 0.94) | 0.039 |
| Symptomatic non-fatal PE | 7/6007 (0.12) | 0.12 | 15/6012 (0.25) | 0.25 | 0.47 (0.19, 1.14) | 0.096 |
| VTE-related death | 43/6007 (0.72) | 0.72 | 46/6012 (0.77) | 0.77 | 0.93 (0.62, 1.42) | 0.751 |
| Death (PE) | 3/6007 (0.05) | 0.05 | 5/6012 (0.08) | 0.08 | 0.60 (0.14, 2.51) | 0.485 |
| Death (PE cannot be ruled out) | 40/6007 (0.67) | 0.67 | 41/6012 (0.69) | 0.69 | 0.98 (0.63, 1.51) | 0.912 |
| Secondary efficacy outcomes | | | | | | |
| VTE-related death | 43/6007 (0.72) | 0.72 | 46/6012 (0.77) | 0.77 | 0.93 (0.62, 1.42) | 0.751 |
| Symptomatic VTE (lower | 11/6007 (0.18) | 0.19 | 25/6012 (0.42) | 0.42 | 0.44 (0.22, 0.89) | 0.023 |
| extremity DVT and non-fatal PE) | | | | | | |
| Composite of symptomatic | 78/6007 (1.30) | 1.31 | 107/6012 (1.80) | 1.80 | 0.73 (0.54, 0.97) | 0.033 |
| VTE and ACM | | | | | | |
| Composite of symptomatic | 94/6007 (1.56) | 1.58 | 120/6012 (2.03) | 2.03 | 0.78 (0.60, 1.02) | 0.073 |
| VTE, MI, Non-hemorrhagic | | | | | | |
| stroke and CV death [3] | | | | | | |
| ACM | 71/60070 (1.18) | 1.19 | 89/6012 (1.49) | 1.49 | 0.80 (0.58, 1.09) | 0.156 |

Note:
ACM = all-cause mortality; CEC = Clinical Event Committee; CI = confidence interval; CrCl = creatinine clearance; CV = cardiovascular; DVT = deep vein thrombosis; MI = myocardial infarction; PE = pulmonary embolism; VTE = venous thromboembolism.
Note:
The primary efficacy outcome is the composite of all symptomatic VTE events (lower extremity DVT, non-fatal PE) and VTE-related death.
Note:
Event Rate 100 pt-45 dy: number of events per 100 subjects in 45 days of follow up. Subjects who do not have events are censored on the minimum of last visit before or on death, or Day 45. For death outcomes, subjects who do not have events are censored on the minimum of last contact date or Day 45.
Note:
Up-to-Day 45 includes all data from randomization to Day 45 (inclusive).
[1] Hazard ratio (95% CI) is from the Cox proportional hazard model, stratified by baseline CrCl (30-<50 mL/min vs. ≥50 mL/min), with treatment as the only covariate.
[2] P-value (2-sided) for superiority of rivaroxaban versus placebo from the Cox proportional hazard model.
[3] CV death includes VTE-related death (PE and PE cannot be ruled out).

Primary Efficacy Outcome—Composite of Symptomatic VTE Events and VTE-Related Death:
The superiority of rivaroxaban over placebo for the primary efficacy outcome was not established. Based on the ITT analysis set, Up-to-Day 45 analysis phase, as adjudicated by the CEC, the number (and incidence) of the first occurrence of the primary efficacy outcome was 50 (0.83%) subjects in the rivaroxaban group compared with 66 (1.10%) subjects in the placebo group, an absolute difference of 0.27% (relative risk reduction [RRR]: 24%; HR: 0.76; 95% CI: 0.52, 1.09; p=0.136).
All preplanned sensitivity analyses showed consistent results for the primary efficacy outcome and the results were generally consistent across subgroups.
Based on the ITT analysis set, Up-to-Day 45 analysis phase, as adjudicated by the CEC:
   In the 10 mg dose stratum, the number (and incidence) of the first occurrence of the primary efficacy outcome was 32 (0.65%) subjects in the rivaroxaban group compared with 48 (0.98%) subjects in the placebo group (RRR: 33%; HR: 0.67; 95% CI: 0.43, 1.04; p=0.075).
   In the 7.5 mg dose stratum, the number (and incidence) of the first occurrence of the primary efficacy outcome was 18 (1.64%) subjects in the rivaroxaban group compared with 18 (1.64%) subjects in the placebo group (RRR: 0%; HR: 1.00; 95% CI: 0.52, 1.92; p=0.994).
A post-hoc sensitivity analysis based on the investigator's assessment showed a nominally significant reduction in the primary efficacy outcome for rivaroxaban compared with placebo. Based on the ITT analysis set, Up-to-Day 45 analysis phase, as reported by the investigator, the number (and incidence) of primary efficacy outcome was 15 (0.25%) subjects in the rivaroxaban group compared with 37 (0.62%) subjects the placebo group, an absolute difference of 0.37% (RRR: 60%; HR: 0.40; 95% CI: 0.22, 0.74; p=0.003).

Secondary Efficacy Outcomes

As the primary efficacy outcome was not statistically significant, the analysis of the secondary efficacy outcomes was considered exploratory and for descriptive purposes only. All p-values were nominal without adjustments for multiple testing. Nominal significance below refers to an alpha level of 0.05 (2-sided).
Based on the ITT analysis set, Up-to-Day 45 analysis phase, as adjudicated by the CEC:
   The number (and incidence) of VTE-related death was 43 (0.72%) subjects in the rivaroxaban group compared with 46 (0.77%) subjects in the placebo group (HR: 0.93; 95% CI: 0.62, 1.42; p=0.751).
   The number (and incidence) of the first occurrence of symptomatic VTE was nominally significantly lower in the rivaroxaban group (11 [0.18%] subjects) than in the placebo group (25 [0.42%] subjects), an absolute difference of 0.24% (RRR: 56%; HR: 0.44; 95% CI: 0.22, 0.89; p=0.023).
   The number (and incidence) of the first occurrence of the composite of symptomatic VTE and ACM was nominally significantly lower in the rivaroxaban group (78 [1.30%] subjects) than in the placebo group (107 [1.78%] subjects), an absolute difference of 0.48% (RRR: 27%; HR: 0.73; 95% CI: 0.54, 0.97; p=0.033).
   The number (and incidence) of the first occurrence of the composite of symptomatic VTE, MI, non-hemorrhagic stroke and CV death was 94 (1.56%) subjects in the rivaroxaban group compared with 120 (2.00%) subjects in the placebo group (HR: 0.78; 95% CI: 0.60, 1.02; p=0.073).
   The number (and incidence) of ACM was 71 (1.18%) subjects in the rivaroxaban group compared with 89 (1.48%) subjects in the placebo group (HR: 0.80; 95% CI: 0.58, 1.09; p=0.156).

Exploratory Efficacy Outcomes

The analysis of exploratory efficacy outcomes was for descriptive purposes only. All p-values were nominal without adjustments for multiple testing. Nominal significance below refers to an alpha level of 0.05 (2-sided).
Based on the ITT analysis set, Up-to-Day 45 analysis phase, as adjudicated by the CEC:
   The number (and incidence) of symptomatic non-fatal PE was 7 (0.12%) subjects in the rivaroxaban group compared with 15 (0.25%) subjects in the placebo group (HR: 0.47; 95% CI: 0.19, 1.14; p=0.096).
   The number (and incidence) of symptomatic lower extremity DVT was nominally significantly lower in the rivaroxaban group (4 [0.07%] subjects) compared with the placebo group (13 [0.22%] subjects), an absolute difference of 0.15% (RRR: 69%; HR: 0.31; 95% CI: 0.10, 0.94; p=0.039).
   The number (and incidence) of symptomatic upper extremity DVT was no subject in the rivaroxaban group compared with 1 (0.02%) subject in the placebo group.
   The number (and incidence) of MI was 15 (0.25%) subjects in the rivaroxaban group compared with 11 (0.18%) subjects in the placebo group (HR: 1.36; 95% CI: 0.63, 2.97; p=0.436).
   The number (and incidence) of non-hemorrhagic stroke was 19 (0.32%) subjects in the rivaroxaban group compared with 30 (0.50%) subjects in the placebo group (HR: 0.63; 95% CI: 0.36, 1.12; p=0.117).
   The number (and incidence) of rehospitalization for symptomatic VTE within 30 days after randomization was 9 (0.15%) subjects in the rivaroxaban group compared with 18 (0.30%) subjects in the placebo group (HR: 0.50; 95% C: 0.22, 1.11; p=0.089).

Pharmacokinetic Results

The dose-adjusted systemic exposure of rivaroxaban was generally comparable for both doses (10 mg and 7.5 mg QD), with a tendency toward slightly lower exposures for the 7.5 mg dose relative to the 10 mg dose. The geometric mean concentrations for subjects receiving the 7.5 mg rivaroxaban dose (baseline CrCl ≥30-<50 mL/min) were approximately 7% to 15% lower than those for subjects receiving the 10 mg dose (baseline CrCl ≥50 mL/min).

Results of the population PK analysis, provided in a separate report, were in concordance with the non-compartmental analysis included in this report. Overall, the population analysis mainly supported the dose adjustment scheme based on baseline CrCl that provided comparable rivaroxaban systemic exposure in subjects with different levels of CrCl.

Medical Resource Utilization and Health Economics Results

The following results were based on the ITT analysis set and Up-to-Day 45 analysis phase:
There were 379 (6.3%) of 6,007 subjects in the rivaroxaban group and 412 (6.9%) of 6,012 subjects in the placebo group with at least 1 re-hospitalization during the Up-to-Day 45 analysis phase. Approximately 90% of these re-hospitalized subjects had a total duration of 3 days in the hospital (91.3% of subjects in the rivaroxaban group and 88.6% of subjects in the placebo group). The mean total duration of all re-hospitalization was 10.8 days for subjects in the rivaroxaban group and 11.0 days for subjects in the placebo group who were re-hospitalized.

Safety Results

Unless otherwise stated, safety analyses presented below were based on the safety analysis set and on-treatment analysis phase. The analysis results of major bleeding (principal safety outcome), composite of major NMCR bleeding (i.e., clinically relevant bleeding), NMCR bleeding, and other bleeding are summarized below.

Hazard Ratio and 95% Confidence Interval for Time to the First Occurrence of Bleeding,
On-Treatment as Adjudicated by the CEC (Study RIVAROXDVT3002: Safety Analysis Set)

| | Rivaroxaban | | Placebo | | Rivaroxaban vs. Placebo | |
|---|---|---|---|---|---|---|
| | | Event Rate | | Event Rate | Hazard Ratio | |
| | n/N (%) | (100 pt-45 dy) | n/N (%) | (100 pt-45 dy) | (95% CI) [1] | p-value [2] |
| Major bleeding | 17/5982 (0.28) | 0.28 | 9/5980 (0.15) | 0.15 | 1.88 (0.84, 4.23) | 0.124 |
| Major and NMCR | 102/5982 (1.71) | 1.71 | 60/5980 (1.00) | 1.01 | 1.70 (1.23, 2.33) | 0.001 |
| bleeding | | | | | | |
| NMCR bleeding | 85/5982 (1.42) | 1.43 | 51/5980 (0.85) | 0.86 | 1.66 (1.17, 2.35) | 0.004 |
| Other bleeding | 54/5982 (0.90) | 0.91 | 34/5980 (0.57) | 0.57 | 1.59 (1.03, 2.44) | 0.035 |

Note:
CEC = Clinical Event Committee; CI = confidence interval; CrCl = creatinine clearance; NMCR = non-major clinically relevant.
Note:
Event Rate 100 pt-45 dy: number of events per 100 subjects in 45 days of follow up. Subjects who did not have events were censored on the minimum of last visit before or on death, or last dose +2 days.
Note:
On-Treatment includes all data from randomization to 2 days after the last dose of the study drug (inclusive).
[1] Hazard ratio (95% CI) is from the Cox proportional hazard model, stratified by baseline CrCl (≥30-<50 mL/min vs. ≥50 mL/min), with treatment as the only covariate.
[2] P-value (two-sided) for superiority of rivaroxaban versus placebo from the Cox proportional hazard model.

Based on the safety analysis set and on-treatment analysis phase, as adjudicated by the CEC:
The number (and incidence) of the first occurrence of major bleeding (principal safety outcome) was 17 (0.28%) subjects in the rivaroxaban group compared with 9 (0.15%) subjects in the placebo group (HR: 1.88; 95% CI: 0.84, 4.23; p=0.124).
   In the 10 mg dose stratum, the number (and incidence) of the first occurrence of major bleeding was 13 (0.27%) subjects in the rivaroxaban group compared with 9 (0.18%) subjects in the placebo group (HR: 1.44; 95% CI: 0.62, 3.37; p=0.398).
   In the 7.5 mg dose stratum, the number (and incidence) of the first occurrence of major bleeding was 4 (0.37%) subjects in the rivaroxaban group compared with no subjects in the placebo group.
Fatal bleeding occurred in 2 (0.03%) subjects in the rivaroxaban group (both in the 10 mg dose stratum, 0.04%) compared with no placebo subject. Major bleeding at a critical site occurred in 3 (0.05%) subjects in the rivaroxaban group and 2 (0.03%) subjects in the placebo group. All critical site bleeding was intracranial in both treatment groups.
The number (and incidence) of the first occurrence of NMCR bleeding was significantly greater in the rivaroxaban group (85 [1.42%] subjects) compared with the placebo group (51 [0.85%] subjects) (HR: 1.66; 95% CI: 1.17, 2.35; p=0.004).
   In the 10 mg dose stratum, the number (and incidence) of the first occurrence of NMCR bleeding was significantly greater in the rivaroxaban group (73 [1.49%] subjects) compared with the placebo group (41 [0.84%] subjects) (HR: 1.78; 95% CI: 1.21, 2.61; p=0.003).
   In the 7.5 mg dose stratum, the number (and incidence) of the first occurrence of NMCR bleeding, on-treatment analysis phase, was 12 (1.10%) subjects in the rivaroxaban group compared with 10 (0.92%) subjects in the placebo group (HR: 1.19; 95% CI: 0.51, 2.76; p=0.684).
The number (and incidence) of the first occurrence of clinically relevant bleeding (ie, composite of major bleeding and NMCR bleeding) was significantly greater in the rivaroxaban group (102 [1.71%] subjects) compared with the placebo group (60 [1.00%] subjects) (HR: 1.70; 95% CI: 1.23, 2.33; p=0.001).
   In the 10 mg dose stratum, the number (and incidence) of the first occurrence of clinically relevant bleeding was significantly greater in the rivaroxaban group (86 [1.76%] subjects) compared with the placebo group (50 [1.02%] subjects) (HR: 1.72; 95% CI: 1.21, 2.43; p=0.002).
   In the 7.5 mg dose stratum, the number (and incidence) of the first occurrence of clinically relevant bleeding was numerically greater in the rivaroxaban group (16 [1.47%] subjects) compared with the placebo group (10 [0.92%] subjects) (HR: 1.59; 95% CI: 0.72, 3.50; p=0.251).
The number (and incidence) of the first occurrence of Other bleeding was significantly greater in the rivaroxaban group (54 [0.90%] subjects) compared with the placebo group (34 [0.57%] subjects) (HR: 1.59; 95% CI: 1.03, 2.44; p=0.035).
   In the 10 mg dose stratum, the number (and incidence) of the first occurrence of Other bleeding was 41 (0.84%) subjects in the rivaroxaban group compared with 28 (0.57%) subjects in the placebo group (HR: 1.46; 95% CI: 0.90, 2.37; p=0.121).
   In the 7.5 mg dose stratum, the number (and incidence) of the first occurrence of Other bleeding was 13 (1.19%) subjects in the rivaroxaban group compared with 6 (0.55%) subjects in the placebo group (HR: 2.16; 95% CI: 0.82, 5.68; p=0.119).
The bleeding results, in general, were consistent across subgroups and with the results of the analysis of primary safety outcome and with that of NMCR bleeding.
More subjects in the rivaroxaban group than in the placebo group discontinued study drug permanently due to bleeding (63 [1.1%] vs 29 [0.5%] subjects, respectively).
   In the 10 mg dose stratum, 52 (1.1%) subjects in the rivaroxaban group discontinued study drug permanently due to bleeding compared with 24 (0.5%) subjects in the placebo group.
   In the 7.5 mg dose stratum, 11 (1.0%) subjects in the rivaroxaban group discontinued study drug permanently due to bleeding compared with 5 (0.5%) subjects in the placebo group.

An overall summary of investigator-reported AEs is provided for the safety analysis set below.

Overview of Adverse Events (Study RIVAROXDVT3002: Safety Analysis Set)

| | Rivaroxaban | Placebo |
|---|---|---|
| | (N = 3982) | (N = 5980) |
| | n (%) | n (%) |
| Any AEs | 1438 (24.0) | 1490 (24.9) |
| Post-baseline AEs | 1430 (23.9) | 1483 (24.8) |
| TEAEs | 1198 (20.0) | 1267 (21.2) |
| AEs with onset >2 days from stop of study drug | 419 (7.0) | 442 (7.4) |
| SAEs | 460 (7.8) | 486 (8.1) |
| Post-baseline SAEs | 465 (7.8) | 485 (8.1) |
| TESAEs | 337 (5.6) | 368 (6.2) |
| SAEs with onset >2 days from stop of study drug | 169 (2.8) | 166 (2.8) |
| Post-baseline AEs leading to permanent study agent discontinuation | 131 (2.2) | 178 (3.0) |
| AEs with outcome of death | 95 (1.6) | 103 (1.7) |

Note:
AE = adverse event;
MedDRA = Medical Dictionary for Regulatory Activities;
SAE = serious adverse event;
TEAE = treatment-emergent adverse event;
TESAE = treatment-emergent serious adverse event.
Note:
Based on MedDRA version 20.1.
Note:
Post-baseline events are defined as those started on or after the first dose of study drug. Treatment-emrgent events are defined as those started on or after the first dose of study drug and up to 2 days after the last dose of study drug.

The proportions of subjects with treatment-emergent adverse events (TEAEs) and treatment-emergent serious adverse events (TESAEs), including those leading to fatal outcomes, were generally balanced between the treatment groups. In both treatment groups, the proportions of subjects with TEAEs or TESAEs were slightly higher for subjects in the 7.5 mg dose stratum than in the 10 mg dose stratum.
The most frequently reported TEAEs in the rivaroxaban group (>1% of subjects) were headache (1.6% vs 2.0% in the placebo group), COPD (1.3% vs 1.3%), and diarrhoea (1.1% vs 0.8%). No TEAE met the threshold of occurring in at least 1% of rivaroxaban subjects and at least 50% more frequently than with placebo.
There were fewer subjects with AEs leading to permanent treatment discontinuation in the rivaroxaban group (131 [2.2%] subjects) than in the placebo group (178 [3.0%] subjects).
There were no new safety signals uncovered for rivaroxaban.

Benefit-Risk Analysis

Although the primary efficacy endpoint was not statistically different between rivaroxaban and placebo, the benefit-risk analyses were conducted to further assess the totality of the data. Based on the ITT analysis set and Up-to-Day 45 analysis phase, for 10,000 subjects treated with rivaroxaban up to 45 days, on average, rivaroxaban would prevent 27 events of the composite of symptomatic VTE and VTE-related death, while causing 12 more major bleeding events than with placebo. The 10 mg dose stratum showed an improved benefit-risk balance, in which rivaroxaban, on average, would prevent 33 events of the composite of symptomatic VTE and VTE-related death, while causing 8 more major bleeding events than with placebo. However, the interpretation of these results could be challenging, as the benefits and risks were comprised of endpoints with different clinical severity, e.g., while the majority of the primary efficacy events were VTE-related death, only a small portion of the between-treatment difference was driven by a difference in VTE-related death, and most of the between-treatment difference in major bleeding was driven by non-fatal and non-critical organ bleeding. Therefore, focusing instead on major thrombotic events that were fatal or caused potentially irreversible harm, on average, rivaroxaban would prevent 55 events of the composite of nonfatal PE, MI, non-hemorrhagic stroke, and non-hemorrhagic ACM, while causing 2 more events of the composite of critical site bleeding and fatal bleeding than with placebo. This benefit-risk profile notably favors rivaroxaban. Similarly, the 10 mg dose stratum showed a better benefit-risk balance, in which rivaroxaban would, on average, prevent 63 events of the composite of nonfatal PE, MI, non-hemorrhagic stroke, and non-hemorrhagic ACM, while causing no additional critical site and fatal bleeding.

These results suggest an overall favorable benefit-risk balance for rivaroxaban compared with placebo, particularly for patients treated with rivaroxaban 10 mg.

Conclusions for Study 2: MARINER

The superiority of rivaroxaban over placebo for the primary efficacy outcome was not established. Rivaroxaban did not significantly reduce the composite primary endpoint of symptomatic VTE (lower extremity DVT and non-fatal PE) and VTE-related death (death due to PE or death in which PE could not be ruled out as the cause) when given prophylactically for 45 days post-hospital discharge to high-risk, medically ill subjects compared with placebo. There was a 24% RRR in the composite primary endpoint of symptomatic VTE and VTE-related death, with an absolute difference of 0.27% compared with placebo.

As the primary endpoint was not achieved, all tests for secondary endpoints in the hierarchy were considered exploratory and p-values were nominal without adjustments for multiple testing. Nominal significance below refers to an alpha level of 0.05 (2-sided).
There appeared to be no effect of rivaroxaban treatment on VTE-related death compared with placebo.
There was a nominally significant 56% RRR in symptomatic VTE, with an absolute difference of 0.24% compared with placebo.
There was a nominally significant 27% RRR in the composite endpoint of symptomatic VTE and ACM, with an absolute difference 0.48% compared with placebo.
There was a trend with numerically fewer events in the rivaroxaban group compared with placebo on the composite endpoint of symptomatic VTE, MI, non-hemorrhagic stroke, and CV death.
There appeared to be no significant effect of rivaroxaban treatment on ACM compared with placebo.
Subjects randomized to rivaroxaban were treated with 10 mg QD, with a dose adjustment to 7.5 mg for subjects with moderate renal insufficiency. Rivaroxaban 10 mg QD had a lower estimated HR and upper bound of the 95% CI than rivaroxaban 7.5 mg QD.
A numerically greater incidence in most bleeding categories was seen in the rivaroxaban group compared with placebo. Rivaroxaban was associated with a statistically significant increase in NMCR bleeding and Other bleeding events compared with placebo. The incidence of major bleeding, major bleeding at a critical site, and fatal bleeding were all generally low with both rivaroxaban dose strata and not significantly different from placebo.
   Major and Other bleeding incidences were numerically higher, while NMCR was significantly higher, for the rivaroxaban 10 mg group compared with the placebo group.
   Major, NMCR, and Other bleeding incidences were numerically higher, but not significantly different, for the rivaroxaban 7.5 mg group compared with the placebo group. The small number of events in this subpopulation precludes any definitive conclusions.
A larger proportion of subjects in the rivaroxaban group (1.1%) discontinued study drug permanently due to bleeding than subjects in the placebo group (0.5%), primarily because of NMCR bleeding (0.7% of rivaroxaban subjects and 0.3% of placebo subjects).
The benefit-risk analyses showed that treatment with rivaroxaban would prevent more harmful events (i.e., efficacy outcome events) than it would cause (i.e., bleeding events). These results suggest an overall favorable benefit-risk balance for rivaroxaban compared with placebo. This was especially true when focused on major thrombotic events that were fatal or caused potentially irreversible harm compared with critical site or fatal bleeding, particularly for subjects treated with rivaroxaban 10 mg.
Re-hospitalizations (6.3% of subjects in the rivaroxaban group and 6.9% of subjects in the placebo group) and duration of re-hospitalizations during the Up-to-Day 45 analysis phase were generally balanced between the 2 groups and a mean of 10.8 days and 11.0 days for the rivaroxaban and placebo groups respectively.
No new safety signals were uncovered for rivaroxaban.
Overall, the MARINER study demonstrated that treating an at risk medically ill population at a lower risk of bleeding with 10 mg rivaroxaban daily (with a dose-adjusted strategy for subjects with moderate renal insufficiency) did not significantly reduce symptomatic VTE and VTE-related death compared with placebo but did demonstrate an acceptable safety profile.

Retrospective Study Analysis a Subpopulation of Study 1

Using key exclusion criteria from Study 2: MARINER to exclude subjects with a higher risk of bleeding, a subpopulation of Study 1: MAGELLAN was retrospectively analyzed to re-evaluate benefit-risk in this subpopulation, which included efficacy endpoints of symptomatic VTE and VTE-related death and safety endpoints of major bleeding events. The key exclusion criteria from Study 2: MARINER that were retrospectively applied to the Study 1: MAGELLAN population for this analysis included: active gastroduodenal ulcer within 3 months of randomization/currently symptomatic; any bleeding within 3 months prior to randomization or during index hospitalization; active cancer at randomization; medical history of severe bronchiectasis/pulmonary cavitation; or dual antiplatelet therapy at baseline. Subjects that met one or more of these criteria prior to or at randomization using standardized MedDRA queries or preferred terms of MedDRA version 13.1 based on data entered into the case report forms in the original MAGELLAN database were excluded from the subpopulation analysis. The analysis of this subpopulation represented approximately 80% of the total Study 1: MAGELLAN study population, and demonstrated similar efficacy with lower risk of bleeding.

The retrospective analysis of the subpopulation of Study 1: MAGELLAN demonstrated that efficacy of rivaroxaban was maintained for both protocol-specified co-primary efficacy endpoints and had a favorable benefit-risk profile due to improved safety.

The MAGELLAN study (Study 1) randomized 8,101 patients from 562 sites in 52 countries and the safety population of patients that received at least one dose of study medication was 7,998 patients. The five key risk factors for major bleeding events in MAGELLAN were identified using study data forms as described herein. With the retrospective use of these risk factors, 1,551 patients were excluded from MAGELLAN and the remaining 6,447 patients, approximately 80% of MAGELLAN, represented the subpopulation. Patients in both treatment groups had no differences in baseline characteristics, and had similar medical histories, with at least 99% of patients having complete immobility or decreased mobility and median total number of days in hospital of 10.

Efficacy Outcomes in the Subpopulation

At Day 10 (per-protocol analysis set), the incidence of the primary efficacy endpoint outcome in the rivaroxaban group was 2.4% (58/2385) and 3.0% (72/2433) in the enoxaparin group (RR 0.820, 95% CI 0.58-1.15). Thus, rivaroxaban is likely non-inferior to enoxaparin, based on a non-inferiority margin of 1.5 in the original MAGELLAN study. At Day 35 (mITT analysis set), the incidence of the primary efficacy outcome in the rivaroxaban group (3.9% [94/2419]) was significantly lower than in the enoxaparin group (5.7% [143/2506], RR 0.68, 95% CI 0.53-0.88, p=0.0029). Asymptomatic lower extremity proximal DVT was the predominant event in both treatment groups (rivaroxaban, 3.0%; enoxaparin, 4.4%). The incidence of VTE-related death in the rivaroxaban group (0.6%) was numerically lower than the enoxaparin group (1.0%).

Safety Outcomes in the Subpopulation

Through Day 35, the safety improved in the rivaroxaban group compared to enoxaparin. In Study 1: MAGELLAN, the incidence of major bleeding was 1.1% in the rivaroxaban group and 0.4% in the enoxaparin-placebo group (RR 2.87, 95% CI 1.60-5.15). Through Day 35 for the subpopulation, the rivaroxaban group and enoxaparin-placebo had incidence of major bleeding at 0.68% and 0.47%, respectively (RR 1.48, 95% CI 0.77-2.84). The rivaroxaban group had a reduction in relative risk for major bleeding from 2.18 (95% CI 1.07-4.45) in the entire population to 1.19 (95% CI 0.54-2.65) in the subpopulation at Day 10. At Day 35, fatal bleeds with rivaroxaban were reduced from 0.2% in the entire population to less than 0.1% in the subpopulation. While Study 1: MAGELLAN had 5 fatal bleeds (0.1%) with rivaroxaban at Day 10, that number was reduced to 1 (less than 0.1%) in the subpopulation analysis, while there was 1 fatal bleed in the enoxaparin/placebo group for the entire population and subpopulation. There were 2 fatal bleeds for the entire population and subpopulation in the post-discharge phase for the rivaroxaban and 0 in the enoxaparin/placebo groups. At Day 35, the relative risk for clinically relevant bleeding (the composite of major and NMCR bleeding) was similar in the overall MAGELLAN population (4.1% vs 1.7%, RR 2.46, 95% CI 1.85-3.25) and the subpopulation (3.5% vs 1.5%, RR 2.35, 95% CI 1.69-3.26) for the rivaroxaban and enoxaparin groups respectively. Overall, treatment emergent clinically relevant bleeding events remained higher in the rivaroxaban group compared to the enoxaparin-placebo group mainly due to NMCR.

Benefit-Risk Profile of the Subpopulation

The same time period was utilized, preventing double counting for fatal bleeds, with all bleeds through Day 35 included (even if off treatment), and bleeding deaths were not counted as CV deaths. Using the primary endpoint, there would be 131 fewer events in the rivaroxaban group with a number-to-treat to benefit (NNT) of 77 to prevent one event, but this was counterbalanced by causing 51 more major bleeds. The benefit risk profile was improved in the subpopulation, with 182 events prevented and only 18 major bleeds caused, with NNT and number-to-treat to harm (NNH) of 55 and 560, respectively. The other benefit risk pairs demonstrated similar patterns. More ischemic events were prevented than hemorrhagic events caused in the subpopulation in all five pairs of clinically meaningful endpoints which included asymptomatic and symptomatic VTE and VTE-related death, major bleeding, and fatal and irreversible thrombotic and bleed events. For 35 days of treatment, the number need to treat to prevent 1 event ranged from 55 to 481 for the 5 pairs, while the number needed to harm ranged from 455-1,067.

The use of five key risk factors for major bleeding may significantly improve the benefit risk profile of rivaroxaban for both in-hospital and extended thromboprophylaxis in acutely ill medical patients using clinically meaningful endpoints. Major and fatal bleeding rates in the subpopulation were significantly reduced compared to the entire population of Study 1: MAGELLAN, while anticoagulant efficacy was maintained. These findings have implications for both the safety and the benefit-risk profile of extended thromboprophylaxis with DOACs across a broad group of hospitalized, acutely ill medical patients, of whom modeling estimates suggest that up to 25-30% are at least at moderate risk of post-discharge VTE.

When comparing rivaroxaban to the enoxaparin/placebo group, rivaroxaban produced a favorable benefit-risk profile for all five pairs of clinically meaningful endpoints, including asymptomatic and symptomatic VTE and VTE-related death, major bleeding, and fatal and irreversible thrombotic and bleed events, with NNTs ranging from 55 to 481 and NNHs ranging from 455-1,067. The present invention provides a strategy of extended thromboprophylaxis to 31-39 days with rivaroxaban, potentially preventing fatal or irreversible thrombotic events in approximately 12,000 patients annually and preventing symptomatic VTE and VTE-related death, as well as non-fatal and fatal PE in approximately 24,000 patients annually. The present invention has important public health implications when looking at the morbidity, mortality, and healthcare costs associated with hospital acquired VTE worldwide.

The MARINER trial (Study 2) prospectively tested if five key additional exclusionary criteria could improve the benefit-risk profile of rivaroxaban in a post-discharge setting. The incidence of major bleeding was very low and not statistically significant between groups (0.28% versus 0.15%, HR 1.88 (95% CI 0.84-4.23, p=0.124). The incidence of critical site and fatal bleeding was 0.05% and 0.03% in the rivaroxaban group and 0.03% and 0.0% in the placebo group, respectively. Although MARINER did not meet its primary efficacy outcome, exploration of pre-specified secondary outcomes suggested a significant reduction in symptomatic VTE alone and as a composite endpoint with all cause-mortality. Therefore, an improved benefit-risk profile may be obtained for extended thromboprophylaxis by treating patient at risk for VTE while carefully avoiding treating patients at high risk of bleeding.

As disclosed herein, five key risk factors for major bleeding have been identified in an acutely ill medical population that has an indication for thromboprophylaxis. A previously unknown subpopulation was identified using these five key risk factors, and substantially improved the benefit-risk profile for in-hospital and extended thromboprophylaxis with rivaroxaban. The present invention can aid in the identification of acutely ill medical subjects with the greatest likelihood of benefit and the least risk of harm for extended thromboprophylaxis.

Each of the articles and patent publications discussed herein is hereby incorporated by reference in its entirety herein.

What is claimed is:

1. A method of prophylactic treatment against venous thromboembolism (VTE) and VTE-related death during hospitalization and post-hospital discharge in an adult human patient admitted for an acute medical illness and at risk for thromboembolic complications due to moderate or severe restricted mobility and other risks factors for VTE, the method comprising administering rivaroxaban to the adult human patient in an effective amount to prophylactically treat against VTE and VTE-related death during hospitalization and post-hospital discharge in an adult human patient admitted for an acute medical illness and at risk for thromboembolic complications due to moderate or severe restricted mobility and other risks factors for VTE, wherein the rivaroxaban is administered orally in the effective amount of 10 mg once daily, and wherein the adult human patient is not at high risk of bleeding.

2. The method of claim 1, wherein the acute medical illness comprises heart failure, active cancer, acute ischemic stroke, acute infectious and inflammatory disease and acute respiratory insufficiency.

3. The method of claim 1, wherein the acute medical illness comprises COVID-19.

4. The method of claim 1, wherein other risks factors for VTE comprise prolonged immobilization, age 75 or older, history of cancer, history of VTE, history of heart failure, thrombophilia, acute infections disease contributing to hospitalization, and body mass index (BMI) equal to or greater than 35 kg/m².

5. The method of claim 1, wherein risk factors for high risk of bleeding are selected from the list consisting of: active cancer, medical history of bronchiectasis/pulmonary cavitation, dual antiplatelet therapy at baseline, active gastroduodenal ulcer, and any bleeding in the previous three months prior to hospitalization.

6. The method of claim 1, wherein the administering step begins within 72 hours of hospitalization.

7. The method of claim 1, wherein the administering step continues for up to 39 days.

8. A method of prophylactic treatment against venous thromboembolism (VTE) and VTE-related death during hospitalization and post-hospital discharge in an adult human patient admitted for an acute medical illness and at risk for thromboembolic complications due to moderate or severe restricted mobility and other risks factors for VTE, the method comprising administering a product comprising rivaroxaban to the adult human patient in an effective amount to prophylactically treat against VTE and VTE-related death during hospitalization and post-hospital discharge in an adult human patient admitted for an acute medical illness and at risk for thromboembolic complications due to moderate or severe restricted mobility and other risks factors for VTE, wherein the product comprising rivaroxaban is administered orally in an effective amount of 10 mg once daily.

9. The method of claim 8, wherein the acute medical illness comprises heart failure, active cancer, acute ischemic stroke, acute infectious and inflammatory disease and acute respiratory insufficiency.

10. The method of claim 8, wherein the acute medical illness comprises COVID-19.

11. The method of claim 8, wherein other risks factors for VTE comprise prolonged immobilization, age 75 or older, history of cancer, history of VTE, history of heart failure, thrombophilia, acute infections disease contributing to hospitalization, and body mass index (BMI) equal to or greater than 35 kg/m².

12. The method of claim 8, wherein the adult human patient does not have a high risk of bleeding.

13. The method of claim 12, wherein risk factors for high risk of bleeding are selected from the list consisting of: active cancer, medical history of bronchiectasis/pulmonary cavitation, dual antiplatelet therapy at baseline, active gastroduodenal ulcer, and any bleeding in the previous three months prior to hospitalization.

14. The method of claim 8, wherein the administering step begins within 72 hours of hospitalization.

15. The method of claim 8, wherein the administering step continues for up to 39 days.

16. An effective pharmaceutical product for reducing the risk of venous thromboembolism (VTE) and VTE-related death during hospitalization and post-hospital discharge in an adult human patient at low risk of bleeding admitted for an acute medical illness and at risk for thromboembolic complications due to moderate or severe restricted mobility and other risks factors for VTE, wherein the effective pharmaceutical product comprises 10 mg rivaroxaban for once daily oral administration.

17. The effective pharmaceutical product of claim 16, wherein the acute medical illness comprises heart failure, active cancer, acute ischemic stroke, acute infectious and inflammatory disease and acute respiratory insufficiency.

18. The effective pharmaceutical product of claim 16, wherein the acute medical illness comprises COVID-19.

19. The effective pharmaceutical product of claim 16, wherein the other risks factors for VTE comprise prolonged immobilization, age 75 or older, history of cancer, history of VTE, history of heart failure, thrombophilia, acute infections disease contributing to hospitalization, and body mass index (BMI) equal to or greater than 35 kg/m².

20. The effective pharmaceutical product of claim 16, wherein the once daily oral administration is for 31 to 39 days.
